(19) United States
(12) Patent Application Publication
(10) Pub. No.: US 2009/0171297 A1
(43) Pub. Date: Jul. 2, 2009

(54) CONTAINER AND CAP ASSEMBLY

(76) Inventors: Robert Smith, Balmain (AU); Robert William Lachlan Holmes, Auckland (NZ)

Correspondence Address: JUDY JARECKI-BLACK; PH.D., J.D., 3239 SATELLITE BLVD. 3RD FLOOR, DULUTH, GA, 30096, US

(21) Appl. No.: 12/239,394

(22) Filed: Sep. 26, 2008

(30) Foreign Application Priority Data
Sep. 27, 2007 (NZ) 562037

(51) Int. Cl. A61M 5/32; A61M 5/315

(52) U.S. Cl. 604/198; 604/227

(57) ABSTRACT

A substance delivery system may include a substance delivery device, a cap, and a removal device. The system may be designed for ease of use. The substance delivery device may include an elongated member adapted to interface with a body. Substance delivery devices may be designed allowing for a predetermined separation distance between the body of the device and end of an elongated member used to drive substance from the device.

### INCORPORATION BY REFERENCE

This application claims benefit of New Zealand provisional patent application Serial No. 562037 filed Sep. 27, 2007.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

In addition, commonly assigned design patent applications of ROBERT WILLIAM LACHLAN HOLMES previously filed as “SYRINGE CAP” (U.S. Application No. 29/305,650) and “SYRINGE CAP” (U.S. Application No. 29/305,651) are hereby incorporated by reference.

### FIELD OF THE INVENTION

This invention relates to substance delivery devices and/or caps, particularly but not necessarily solely for use in veterinary applications.

### BACKGROUND OF THE INVENTION

Syringes for injecting substances are well known. In general they consist of a barrel with a tip for dispensing a substance, or a connector suitable for receiving a needle, located at one end, and a plunger which contacts and seals with the interior surface of the barrel at the other end. Finger grips extend radially from the surface of the barrel at the end of the barrel which receives the plunger. The plunger is actuated by the thumb pressing against the end of the plunger. When fully actuated the end of the plunger and the finger grips are adjacent one another.

To provide controlled operation of a syringe during the dispensing procedure the syringe must be kept stable. By “stable” is meant having minimal or no unwanted movement during the dispensing procedure.

Of particular interest are syringes used in the veterinary field for the intramammary application of treatments to prevent or control mastitis, for example in dairy cows. These syringes typically contain an antibiotic paste or gel. To administer this material the veterinarian or farmer typically removes the cap from the syringe to expose a narrow tip that can vary in length from approximately 5 mm to 15 mm. While holding the teat of the cow in one hand and the syringe in the other, the tip of the syringe is inserted into the teat opening. The plunger of the syringe is depressed with the thumb, finger or base of hand. When the contents of the syringe have been fully dispensed the tip is withdrawn and the syringe discarded.

Survey studies in New Zealand have shown that there are a number of methods employed by users to both remove the cap of the syringe and to hold the syringe and dispense the contents.

It was found that: 23.5% of users gripped the cap in their teeth in order to remove it; 14.6% of users pushed the cap off with their thumb in a sideways movement; and 61% of users held the syringe in one hand while pulling the cap off with the other. All users expressed frustration with existing syringe designs because due to the nature of the environment in which they are applied the caps are very slippery to grip. This makes them very difficult to remove.

Users also expressed frustration with the difficulty with which syringes could be held during use. 85% of users held the syringe in what could be classified as the traditional manner with their fingers on the finger grips and thumb on the plunger. 10% of users held the syringe with their fingers on the finger grips and the base of the plunger in the palm of their hand. In the case of intramammary syringes where the cow may be moving around it is important that the syringe can be held firmly at all times. Another particular reason for this is that it is critical that no contamination or bacteria is introduced onto the tip of the syringe during the dispensing procedure. If the syringe is not able to be held firmly it may brush the outside of the udder or the cow's leg or other barrel part, thereby causing contamination of the syringe.

Prior art syringe arrangements do not allow for controlled operation of a syringe during the dispensing procedure, due to the relative positions of the finger grips and the end of the plunger when the syringe is fully actuated. As they are adjacent one another, the dispensing end of the barrel (which may or may not be fitted with a needle) is unsupported during the operation of the syringe, leaving the syringe unstable. If the syringe is unstable, it is difficult to obtain and maintain the correct injection site and this may cause discomfort to the recipient of the injection, together with the problems mentioned above.

Keeping a syringe stable also assists the user in injecting the correct amount of the substance.

In addition, prior art syringes are not ideal for dispensing substances such as viscous pastes and the like. This is because the thumb acting on the plunger has to provide sufficient force to move the plunger down towards the dispensing end of the barrel; an operation which can be inefficient and uncomfortable.

There is a need for a syringe that is more stable than prior art syringes during the injection procedure and which is efficient, comfortable and easy to use irrespective of the substance being administered.

Syringe caps which are currently in use are generally designed to be pulled or pushed or severed off the tip of the syringe prior to use. Accordingly, they can be difficult and tiresome to remove, particularly when using syringes repeatedly during farming operations. Usually they are pushed off with the thumb prior to use, which can cause damage to the syringe tip (bending or breakage) and can also present a contamination risk as the user's thumb or fingers may come into contact with the syringe tip during removal.

The discussion herein of the background to the invention is included to explain the context of the invention. This is not to be taken as an admission that any of the material referred to was published, known or part of the common general knowledge as of the priority date of any of the claims of the invention.

### SUMMARY OF THE INVENTION

In an embodiment, a substance delivery device may include an elongated member adapted to interface with a body of the substance delivery device. Substance delivery devices may include, but are not limited to pumps, syringes, and/or any device currently known or yet to be discovered in the art. A body may include, but is not limited to a barrel, a tube, and/or any device currently known or yet to be discovered in the art. An elongated member may include, but is not limited to a plunger, a piston, and/or any device currently known or yet to be discovered in the art.

The body may define a conduit therethrough. One or more openings may be disposed at the dispensing end of the body. Further, the substance delivery device may include a gripping section which allows a user to grip the body with one or more digits of a user's hand. The gripping section may be positioned at any point along the length of the body. Some embodiments may be configured to maintain a predetermined separation distance between the gripping section and the rear end of the elongated member. Alternatively, some embodiments may be configured to maintain a separation distance between an end of the body and an end of the elongated member. Additionally, the body of the substance delivery device may have a preselected overall length.

In an embodiment, the elongated member may include a seal assembly. The elongated member may be adapted to be reciprocated through a predefined stroke length to move the seal assembly through the conduit within the body. In some embodiments, the seal assembly may be moved from a fully retracted position to a fully depressed position to drive a substance present in the body out an opening positioned in the dispensing end of the body. The elongated member may be moved by applying pressure to an engaging member disposed at a rear end of the engaging member. Pressure may be applied to the engaging member with any surface, including but not limited to a digit or heel of the user's hands.

In some embodiments, the elongated member has a length that is substantially 110% or greater than the preselected overall length of the body. Thus, the engaging member may extend beyond an end of the body a predetermined distance when the elongated member is in the fully depressed position.

In an embodiment, the elongated member has a length that is greater than about 120% of the preselected overall length of the body. In some embodiments, the length of the elongated member may be greater than about 130%, 150%, 170%, 190%, 210%, 230%, 250%, 270% or 290% of the preselected overall length of the body.

In an embodiment, a portion of the elongated member proximate the engaging member is enlarged with respect to the rest of the elongated member for increased stability during use. For example, the elongated member may have a flared end or a tapered outer periphery such that the diameter of the elongated member increases along the length of the elongated member towards the engaging member.

In an embodiment, the engaging member has a larger diameter than the rest of the elongated member for ease of use. The engaging member may be slightly concaved to accommodate application of pressure by the user comfortably during use.

In a further embodiment, the invention may include a substance delivery device having a dispensing end and a rear end located opposite the dispensing end. The substance delivery device may include a body defining a conduit. The body may include openings disposed at the dispensing end of the substance delivery device and a gripping section which allows a user to grip the body. For example, the user may grip the body of the substance delivery device with one or more digits of a user's hand.

The substance delivery device may include an elongated member having a seal assembly. The elongated member may be adapted to be reciprocated through a predefined stroke length to move the seal assembly through the conduit in the body. The elongated member may move from a fully retracted position to a fully depressed position. This movement may drive a substance when present in the body out of the body through the opening. The elongated member may have an engaging member at the rear end of the substance delivery device. A user may apply pressure to the engaging in order to reciprocate the elongated member.

In some embodiments, when the elongated member is in the fully depressed position, the engaging member and the gripping section are spaced from one another by a predetermined separation distance. Alternatively, some embodiments may be configured to maintain a separation distance between an end of the body and an end of the elongated member when the elongated member is in the fully depressed position.

In an embodiment, the separation distance is at least 50% of the predefined stroke length. Some embodiments include a separation distance that is, for example, greater than about 60%, 80%, 100%, 150%, 200% or 250% of the predefined stroke length.

In an embodiment, the separation distance is at least 2 cm. The separation distance may be, for example, greater than about 3 cm, 4 cm, 5 cm, 6 cm, 7 cm, 8 cm, 9 cm or 10 cm.

In an embodiment, the elongated member has a length that is at least 120% of the predefined stroke length. The length of the elongated member may be even greater. For example, some embodiments may include an elongated member having a length that is greater than about 150%, 200%, 250%, 300%, 350% or 400% of the predefined stroke length. In some embodiments, when the elongated member is in the fully depressed position, the engaging member is disposed outward from the body. Thus, the engaging member may be positioned such that the engaging member extends beyond the gripping section by a predetermined separation distance.

In an embodiment, the substance delivery device includes a cap, which is adapted to removably couple to the body to protect the opening when not in use. In some embodiments, a retaining assembly may be disposed on the cap and/or the body.

An embodiment may include a cap adapted to removably couple to a container having an opening to protect the opening when not in use. In some embodiments, a retaining assembly may be disposed on the cap and/or the body.

The cap may include a receiving portion which receives at least a portion of the container therein in an insertion direction. The receiving portion may have a retaining assembly adapted to removably grip the container when present in the receiving portion with a retaining force adapted to removably retain the cap to the container.

Further, a leveragable portion may allow the cap to be removed from the container. The leveragable portion may have any geometry. For example, a leveragable portion may include a shape protruding from the cap body to allow for leverage applied there against allowing for the removal of the cap from the container. In some embodiments, the leveragable portion may include an outwardly contoured portion. Alternatively, the leveragable portion may include a frusto-conical shape in a side view. In an embodiment, the leveragable portion may include a disc shaped member in a side view which can be located at a proximal end or distal end of the cap.

In an embodiment, the container is a substance delivery device and the cap has a top portion adapted to provide a stopper to prevent the tip of the substance delivery device from protruding there from. The top portion may be enclosed.

In still a further aspect, the invention may include a cap removal device adapted to remove a cap from a container. A cap removal device may include a support portion adapted to be supported and to support a cantilever portion. A cantilever portion may be adapted to allow the cap to be removed from the container. In some embodiments, the cantilever portion in combination with the support portion may form a first receiving recess. Some embodiments include a cantilever portion having two prongs forming a second recess therebetween. In such embodiments, when the leveragable portion of the cap is inserted in the first receiving recess and the hollow container receiving portion is inserted in the second recess, the cap can be removed from the container.

In some embodiments, the support portion may be supported by means of a band member adapted to be attached to a wrist, belt, structural element including, but not limited to a wall, a beam, a fence post, a door, a gate and/or any other object. Alternatively, the support portion may be adapted to be hung from an upstand or another structural element.

### BRIEF DESCRIPTION OF THE DRAWINGS

Throughout the body of this description reference will be made to the accompanying drawings in which:

FIG. 1 is an isometric view of the embodiment of the substance delivery device,

FIG. 2 is a perspective view of a substance delivery device body in accordance with an embodiment of the present invention,

FIG. 3 is a top view of the embodiment of the substance delivery device as shown in FIG. 1,

FIG. 4 is a perspective view of a substance delivery device elongated member in accordance with an embodiment of the present invention,

FIG. 5 is a front view of a substance delivery device in a fully depressed position in accordance with an embodiment of the present invention,

FIG. 6 is a cross-sectional view of the embodiment of the substance delivery device as shown in FIG. 5, along the line X-X′,

FIG. 7 is a side view of the embodiment of the substance delivery device as shown in FIG. 5,

FIG. 8 is a front view of a substance delivery device (including a cap) in a fully retracted position in accordance with an embodiment of the present invention,

FIG. 9 is a cross-sectional view of the embodiment of the substance delivery device as shown in FIG. 8, along the line Y-Y′,

FIG. 10 is a side view of the embodiment of the substance delivery device as shown in FIG. 1,

FIG. 11 is a perspective view of the embodiment of the substance delivery device shown in FIG. 1,

FIG. 12 is a perspective view of an unassembled cap and substance delivery device including the body and elongated member,

FIG. 13 is a perspective view of the cap, body and elongated member shown in FIG. 13 as assembled,

FIGS. 14a-e are a series of views of a cap including a cross-sectional view of the embodiment of the cap as shown in FIG. 14a, along the line Z-Z′,

FIG. 15 is a side view of a substance delivery device with a flared cap,

FIG. 16 is a side view of a substance delivery device with a cap having a frusto-conical shaped leveragable portion,

FIG. 17 is a side view of a substance delivery device with a cap having a leveragable portion with a disc-shaped end,

FIG. 18 is a perspective view showing the cap being removed from the substance delivery device by a user,

FIG. 19 is a perspective view of an embodiment of a cap removal device adapted to be worn around a wrist of a user, and

FIG. 20 is a perspective view of a further embodiment of a cap removal device, adapted to be worn on a belt of a user.

### DETAILED DESCRIPTION

The following description is given by way of illustration only and shall not be taken as being in any way limiting as to the spirit or scope of the invention. Referring to the drawings, like numerals designate corresponding parts where possible throughout the several figures.

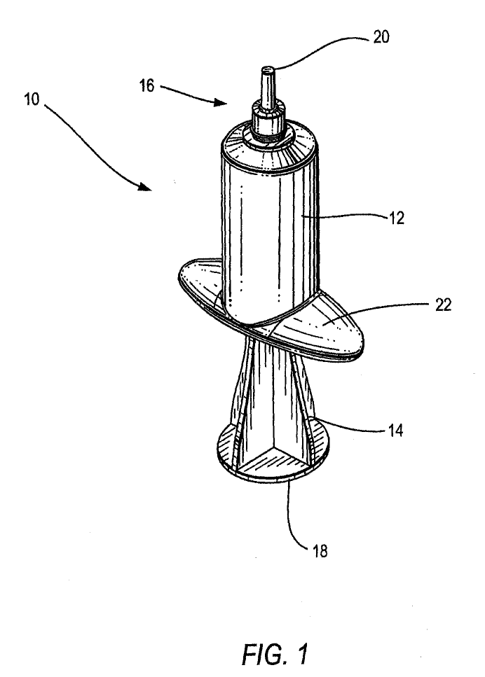

An embodiment of substance delivery device is depicted in FIG. 1. Substance delivery device 10 may include body 12 and elongated member 14. Elongated member 14 may be positioned within body 12 such that dispensing end 16 of the body and rear end 18 of the elongated member are located opposite ends of the substance delivery device.

Body 12 may include opening 20 at dispensing end 16 of substance delivery device 10. In some embodiments, multiple openings may be disposed at the end of the substance delivery device. Alternately, some embodiments may include one or more openings proximate dispensing end 16. Openings may be utilized for dispensing substances including but not limited to medicines. In some embodiments, fluids, including but not limited to viscous fluids such as pastes, gels may be dispensed using the substance delivery device. In some embodiments, dispensing end 16 and/or opening 20 may include a needle. In some embodiments, the dispensing end of the substance delivery device may include a needle, such as a hypodermic needle, a nozzle, and/or tubing to help direct the flow into and out of the body.

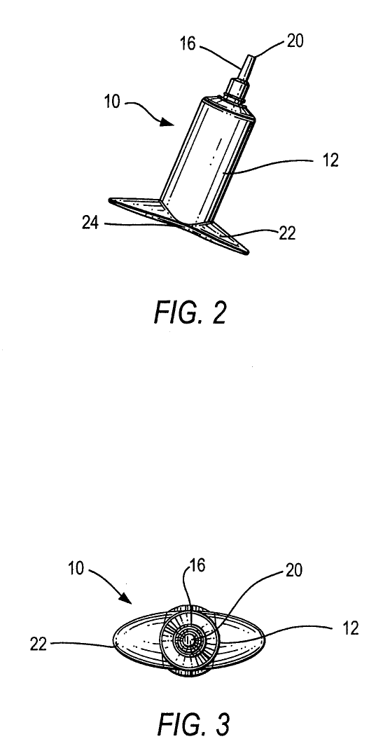

As shown in FIG. 2, body 12 includes gripping section 22 to allow a user to grip the body 12. For example, user may use one or more digits of user's hand. Gripping section 22 may be disposed on body 12 such that the fingers can support substance delivery device 10. Gripping section 22 may assist in obtaining the correct administration site and keeping the substance delivery device substantially stable during operation. In the embodiment shown in FIG. 2, gripping section 22 includes two protrusions. In an embodiment, gripping section 22 may include a pair of opposing projections which extend at an angle from the surface of the body 12 as shown in FIG. 3. In some embodiments, the gripping section may extend substantially radially outward from the body of the substance delivery device. For example, a gripping section may include a protrusion including, but not limited to a flange, rim, collar and/or ring. In some embodiments, the gripping section may be ergonomically designed to accommodate a user's digits and/or hands comfortably. For example, a gripping section may have indentations or curves designed to conform with the shape of a hand or fingers. Use of fingers and/or a hand may support and guide the substance delivery device more effectively during operation due to the design of the substance delivery device. Accordingly, substances may be administered more accurately. In some embodiments, having the ability to activate the substance delivery device by the heel of the hand, may make the operation of the substance delivery device more comfortable to the user.

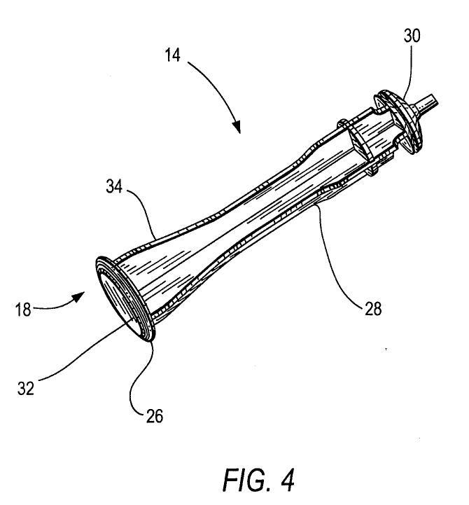

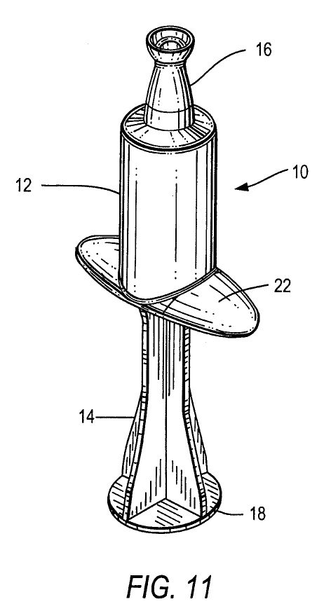

Body 12 is adapted to receive elongated member 14 at end 24 of the body as shown in FIG. 2. Elongated member 14 depicted in FIG. 4 may include engagement section 26, elongated section 28, and seal assembly 30. As shown in FIGS. 3-10, the diameter of elongated member 14 may vary along the length of the elongated member. Thus, the elongated member may have one or more sections which flare outward. Alternately, the elongated member may have sections in which the cross-sectional diameter increases or decreases.

As shown in FIG. 4, the elongated member 14 has an engaging member 32 disposed at rear end 18 for engaging with a digit or heel of a user's hand as the user reciprocates the elongated member 14. In some embodiments, the engaging member 32 may be enlarged so that it can be comfortably engaged with the heel of the user's hand during use. For example, engaging member 32 may have a larger diameter than the rest of elongated member 14. Engaging member 32 may have geometry conducive to applying pressure. In some embodiments, engaging member 32 may be slightly concaved to accommodate the user's thumb, fingers and/or hand comfortably during use.

It will be obvious to those skilled in the art that the engaging member does not have to be operated by the heel of a hand, as the thumb or another digit may be used instead. In some embodiments, using the heel of the hand to depress the engaging member into the body towards the dispensing end may provide increased pressure, and is thus may make it easier and more comfortable to use. In the case of farming operations, ease of use may be valuable where substance delivery devices are used repeatedly for an extended period of time.

As shown in FIG. 4, elongated member may include flared section 34 proximate the engaging member 32. Flared section 34 may provide increased stability during use. In some embodiments, the diameter of the elongated member may vary along a length of the elongated member. Alternatively, an elongated member may have a flared end. In some embodiments, elongated member 14 may have a tapered outer periphery such that the diameter of the elongated member increases along the length of the elongated member towards the engaging member.

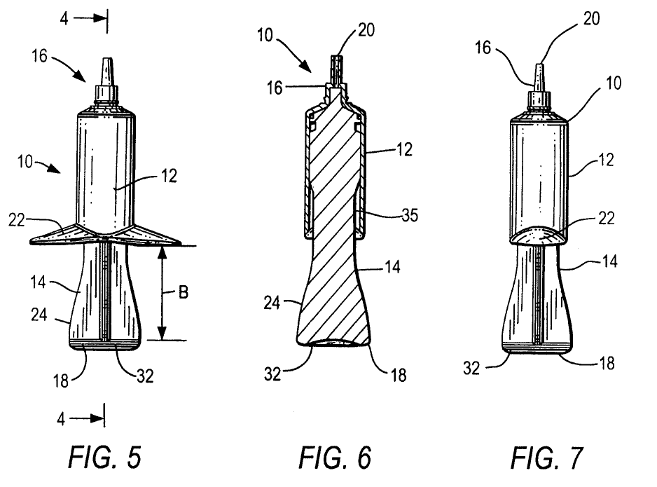

FIGS. 5 to 7 depict elongated member 14 positioned within body 12 in the fully depressed position. As shown in FIGS. 5 to 7, engaging member 32 and gripping section 22 may be spaced from one another by a predetermined separation distance (“B”) (see FIG. 5). As shown in the cross-sectional depiction of FIG. 6, when fully depressed the configuration of the elongated member may maintain the predetermined separation distance (“B”) between engaging member 32 and gripping section 22.

Elongated member 14 may be positioned within conduit 36. In some embodiments, a profile of the elongated member may substantially conform to a profile of the conduit. In alternate embodiments, the profile of the elongated member may conform to portions of the conduit as depicted in FIG. 6. Conduit 36 communicates with opening 20.

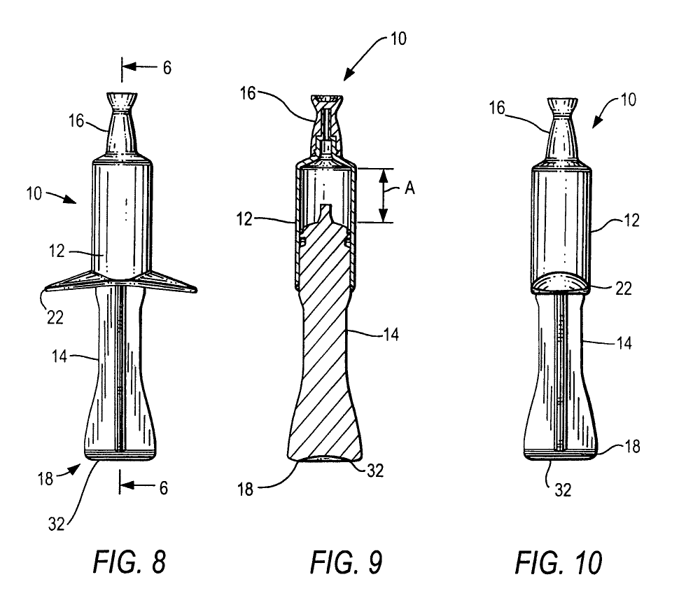

FIGS. 8-10 depict the elongated member positioned within the body. FIG. 8 shows elongated member 32 positioned within body 12.

As shown in FIG. 6 and FIG. 9, elongated member 14 is adapted to be reciprocated through a predefined stroke length to move seal assembly 30 through the internal volume of body 12 from a fully retracted position (See FIG. 9) to a fully depressed position (See FIG. 6) to drive a substance when present in body 12 through opening 20. The predefined stroke length (“A”) (See FIG. 9) may be the distance elongated member 14 is moved from the fully retracted position as shown in FIG. 9 to the fully depressed position as shown in FIG. 6.

A stroke length may be defined as the distance indicated as “A” in FIG. 9. In some embodiments, the stroke length may be predefined. In some embodiments, a separation distance (“B”) (as shown in FIG. 5) may be equal to the value of at least 50% of a predefined stroke length (“A”) (as shown in FIG. 9).

In an embodiment, the separation distance (“B”) may be even greater. In some embodiments, a separation distance may be greater than about 60%, 80%, 100%, 150%, 200% or 250% of the predefined stroke length. For example, the separation distance (“B”) may be at least 2 cm. In alternate embodiments, the separation distance (“B”) may be greater than that. For example, a separation distance (“B”) may be greater than about 3 cm, 4 cm, 5 cm, 6 cm, 7 cm, 8 cm, 9 cm or 10 cm, depending on the desired overall dimensions of the substance delivery device. Alternately, in some embodiments the stroke length may vary.

In an embodiment, the separation distance (“B”) (See FIG. 5) may be achieved by providing elongated member 14 which has a length that is at least 120% of the predefined stroke length (“A”) (See FIG. 9). In an embodiment, the length of the elongated member may be even greater, such as for example, greater than about 150%, 200%, 250%, 300%, 350% or 400% of the predefined stroke length. Accordingly, when the elongated member 14 is in the fully depressed position, the engaging member 32 is disposed outward from the body 12 to result in the engaging member 32 being disposed away from gripping section 22 by the predetermined separation distance (“B”) as shown in FIG. 5.

Described in another way, the elongated member may have a length that is substantially 110% or greater than the overall length of the body (which is preselected depending on the desired overall dimensions of the substance delivery device), such that the engaging member is disposed outward from an end of the body a predetermined distance when the elongated member is disposed in the fully depressed position. In some embodiments, the elongated member has a length that is greater than about 120% of the preselected overall length of the body. For example, the elongated member may have a length that is greater than about 130%, 150%, 170%, 190%, 210%, 230%, 250%, 270% or 290% of the preselected overall length of the body.

In some embodiments, the substance delivery device may include a cap. Caps may be designed to be easily removed prior to use such that removal of the cap does not cause damage and/or contamination to the dispensing end of the syringe. Caps may be removable, reusable and/or configured for single use.

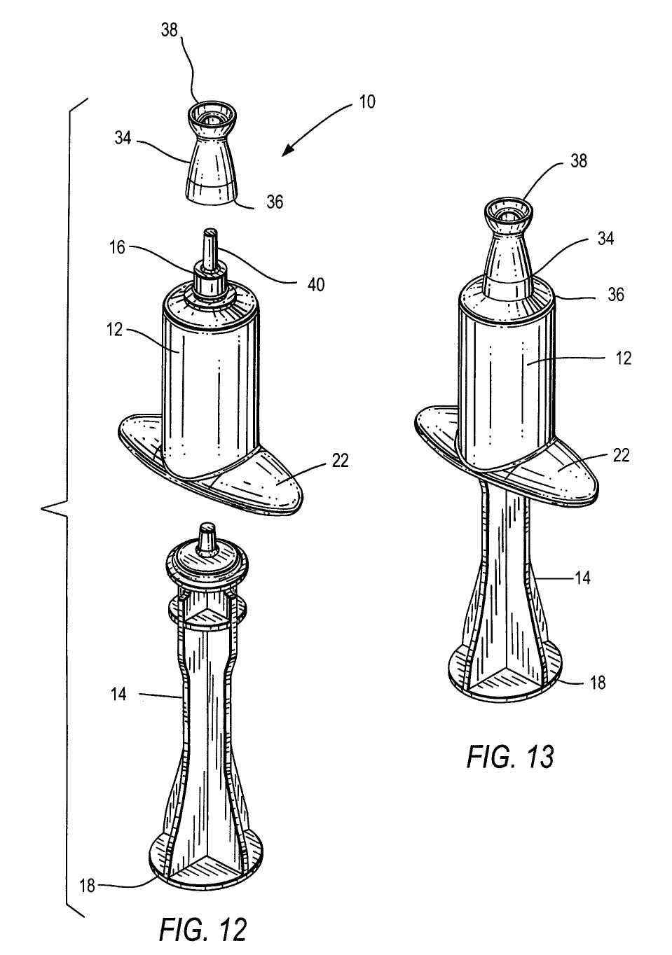

FIGS. 8 to 17 show various embodiments of caps 34 which may be used to protect the tips of substance delivery devices. FIG. 12 shows the substance delivery device assembly comprising of elongated member 14, body 12 and cap 34 in an unassembled form. As shown in FIG. 12, each cap has proximal end 36 which will be positioned closest to body 12 and a distal end 38 positioned near the tip 40 when the cap is in place. In some embodiments, cap 34 may be configured to conform to profile of tip 40. FIG. 13 shows the substance delivery device and cap as assembled and ready to use.

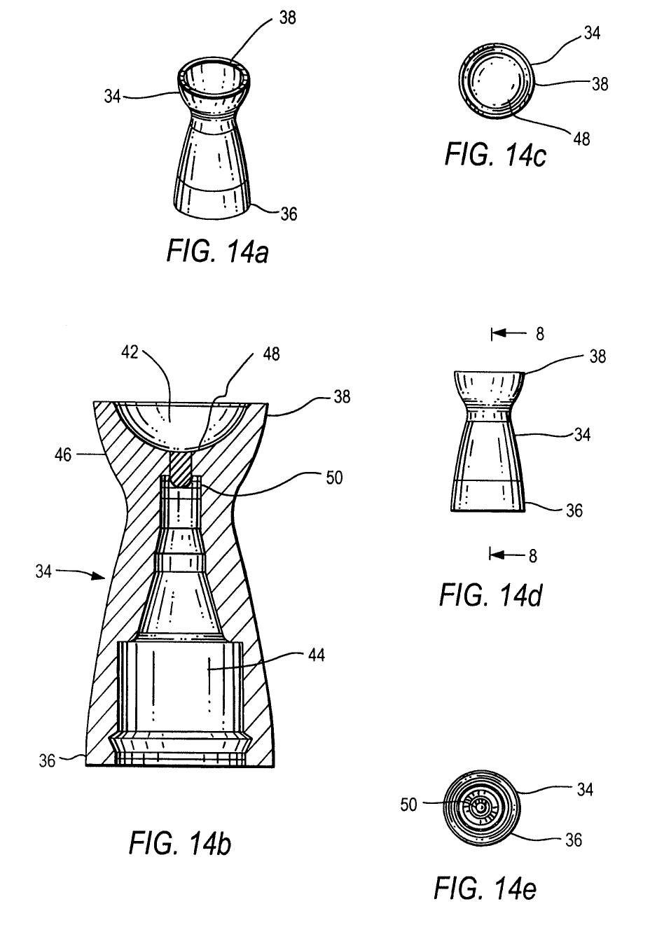

FIG. 14 depicts various views of cap 34. FIG. 14a depicts a perspective view of cap 34. As shown in FIG. 14a, cap 34 may be contoured. Cap 34 has proximal end 36 closest to body 12 and includes a substantially hollow and substantially outwardly contoured, elongate structure. The elongate structure may define a length and have closed surface 42 at distal end 38. FIG. 14b shows a cross-sectional view of the cap depicted in FIG. 14d along line Z-Z′. Receiving portion 44 is coupled to top portion 46. As shown in FIGS. 14b and 14e, each cap has a hollow receiving portion at the proximal end 36 for receiving the substance delivery device tip, and a top portion at the distal end 38 which may provide the means for enabling removal of the cap from the substance delivery device. The top portion may include a stop to prevent the tip of the substance delivery device from protruding therefrom. The stop can include any planar member with or without a recess or recesses. The stop may be curved therein. In one embodiment the stop may include a circumferential rim. Top portion 46 may include an end stop. End stops may include, but are not limited to various surface profiles such as flush, recessed, planar, projections and/or curved surfaces. In addition, end stops may include outer stopping surface 48 and/or inner stopping surface 50. For example, as shown in FIG. 14b inner stopping surface 50 is oriented substantially at right angles to the length of the substance delivery device and cap. In some embodiments, inner stopping surface 50 may engage tip 40 of substance delivery device as shown in FIG. 9.

A receiving portion of a cap may have an internal shape that allows the cap to couple to the substance delivery device. For example, as shown in FIG. 14b receiving portion 44 of cap 34 includes an inner portion shaped in a complementary shape to the substance delivery device outlet. In some embodiments, the cap may be removably held by a retaining assembly (not shown) in place over the tip of the substance delivery device.

In some embodiments, a portion of the cap may removably grip a portion of the substance delivery device. Alternately, a portion of the substance delivery device may be configured to grip a portion of the cap. The receiving portion of the cap may include a retaining assembly adapted to removably grip the substance delivery device when present in the receiving portion with a retaining force adapted to removably retain the cap to the substance delivery device. The retaining assembly may include any suitable configuration that allows for easy removal of the cap in use, yet sufficient cap retention when not in use. For example, retaining assembly may include, but are not limited to fastening mechanisms, friction fits, interference fits, press fits, click in place type mechanisms, and/or any other mechanism currently known or yet to be discovered in the art.

In an embodiment, the cap may protect the substance delivery device and the contents of the substance delivery device from contamination. Further, the cap may protect the user of the substance delivery device. In use, when fitted to a substance delivery device, the proximal end of the cap may abut the body of the substance delivery device.

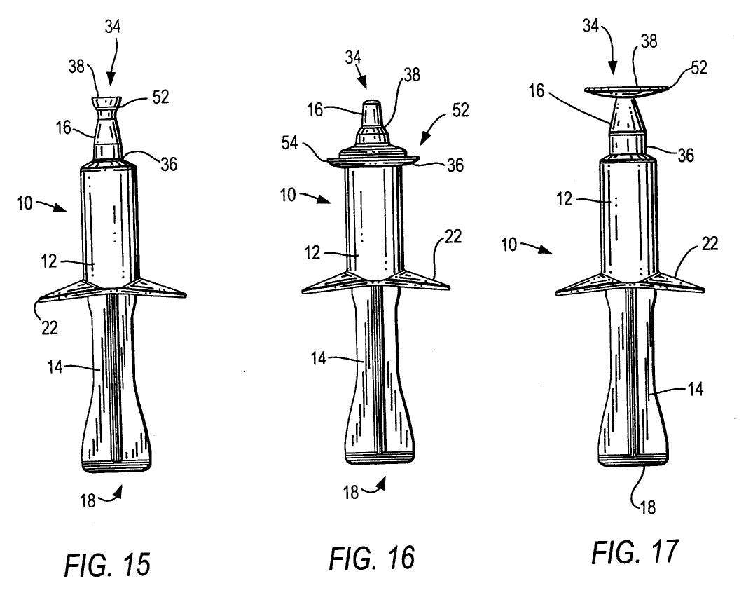

FIGS. 15-17 show various embodiments of cap 34 different profiles having leveragable portion 52. In some embodiments, leveragable portion 52 may be ergonomically shaped to enable a user to easily grip the cap and remove it. In addition, a leveragable portion may include, but are not limited flanges, rims, projections, rings, and/or collars.

FIG. 15 depicts a contoured cap having an ergonomic shape. In addition, the cap substantially conforms to the shape of the substance delivery device tip. In FIG. 16, cap 34 outwardly and inwardly substantially mirrors the shape of the substance delivery device tip. FIG. 16 includes cap 34 having flared rim 54 at the proximal end 36. In FIG. 17, cap 34 has leveragable portion 52 at the distal end 38 which is disc-shaped to enable a user to easily grip the cap so it can be removed from the substance delivery device.

In use, if the substance delivery device is fitted with a cap as herein described, then the cap is first removed from the substance delivery device. The substance delivery device is then gripped using the fingers and the heel of the hand. The heel on the palm of a hand activates the engaging member by pushing it towards the dispensing end of the substance delivery device while the user simultaneously grips the flanges of the gripping section with their fingers.

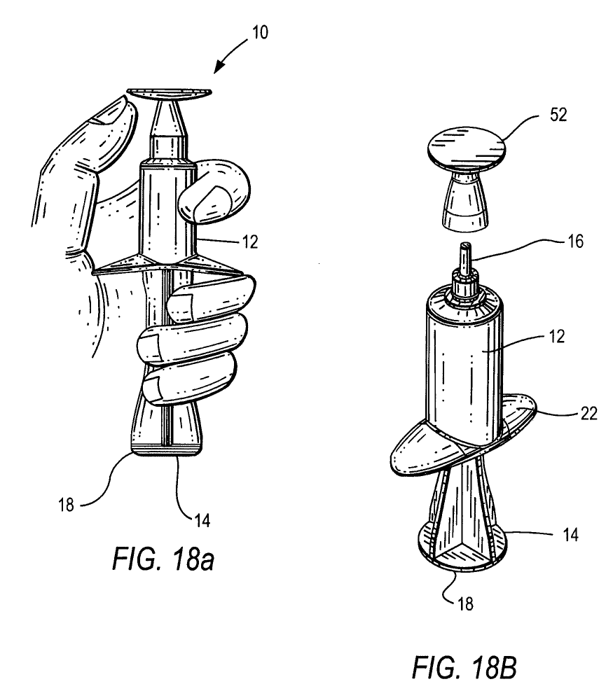

In order to remove any of the caps described herein, a user may simply grip the substance delivery device body and use their thumb to remove the cap, in a simple ‘flicking’ motion, as shown in FIG. 18.

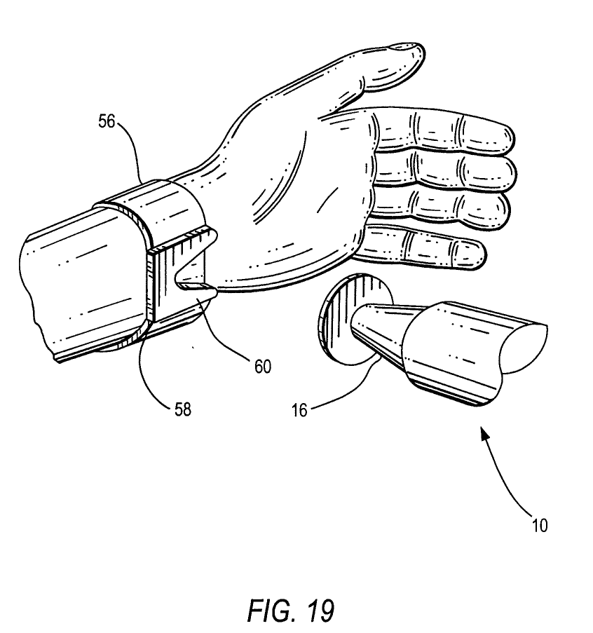

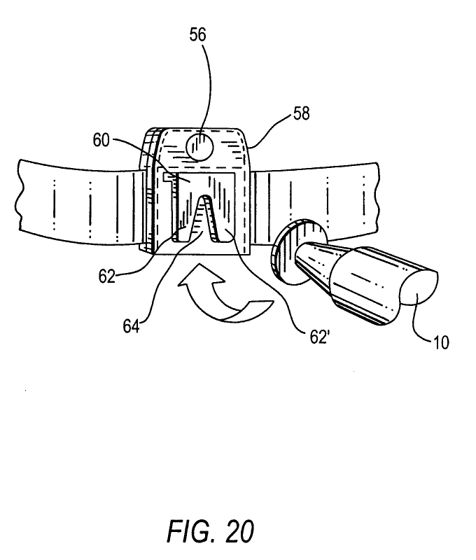

FIGS. 19 and 20 depict alternate embodiments utilizing removal devices 56. The cap removal devices comprise support portion 58 and a cantilever portion 60 having a space or a first receiving recess therebetween. Support portion 58 can be for example, a band or strap which can be worn around a user's wrist (as shown in FIG. 19), or a band or sleeve which can be slidably attached to a user's belt (as shown in FIG. 20). Alternatively, support portion 58 could be adapted to be attached to another object or an upstand or the like. For example, the support portion may include, but is not limited to a hook shaped member and/or a member that is coupled using a fastener to a surface such as, a fence post, wall, framing, and/or any other convenient edge.

Cantilever portion 60 is shaped in such a way that any cap may be inserted therein whilst still attached to a substance delivery device, to enable removal of the cap. As shown in FIG. 20, cantilever portion 60 includes two spaced apart prongs 62, 62′ with recess 64 therebetween. The prongs are spaced from support portion 58 to define a first receiving recess to allow the leveragable portion of cap 34 to be inserted behind the prongs and abut support portion 58 such that at least part of the body of the cap is accommodated in recess 64 between prongs 62, 62′. Once the leveragable portion of the cap is in this position the user only then needs to pull in the direction parallel to the length of the substance delivery device, to remove the cap.

As shown in FIGS. 19 and 20, the method of removing a cap from a container such as a substance delivery device, utilizing a cap removal device as described herein, includes supporting the support portion 58 of the cap removal device by attaching it to a wrist, belt, or other object, inserting the leveragable portion of the cap into the first receiving recess of the cap removal device such that the hollow receiving portion of the cap is accommodated by the second recess, pulling the container/cap assembly until the cap is removed from the container or substance delivery device.

The substance delivery device may provide stability during the injection procedure and allow for controlled administration. When additional stability is provided, the injection procedure may be more effective and there may be less risk of contamination. In addition, activating the elongated member by engaging the heel of the hand with the engaging member may create more pressure and result in easier and less tiresome operation of the substance delivery device. This may be particularly useful when substance delivery devices are being used repeatedly for extended time periods. In some embodiments, the substance delivery device may be designed to be comfortable and easy to use.

In addition, a cap and cap removal device is provided which requires less effort to remove the cap from a container or substance delivery device, and which results in less hand fatigue during repeated operations. Both the design of the cap and/or the use of the cap removal device provides for easier removal of the cap in cases where the cap is slippery. Also, there is less risk that the user's hands or fingers will contact the dispensing tip of the substance delivery device body or other container and cause contamination or damage (such as bending or breakage) to the dispensing tip.

A substance delivery system for use in veterinary field, for example for the intramammary application of treatments, may include any combination of the parts described herein.

Various alterations can be made to the invention as described without departing from the spirit or scope of the invention. For example, as mentioned above, the cap is not restricted to a cap which fits on to a substance delivery device, as such a cap could be designed for other types of containers having openings which require protection.

The measurements as exemplified herein are given by way of example only and are of course dependent on the overall dimensions of the substance delivery device and are therefore variable. The measurements are given to reflect a substance delivery device that could be reasonably used by a user with one hand. In some embodiments, the substance delivery device may be used with the fingers and the heel of the hand simultaneously.

The invention is in no way limited to the embodiments described herein as they are purely to exemplify the invention only and possible variations and modifications which would be readily apparent are intended to be included within the scope of the invention.

What is claimed is:

1. A substance delivery system comprising:
a body having a conduit therethrough comprising:
an opening disposed at a dispensing end of the body;
a gripping section disposed on the body; and
a elongated member having a diameter varied along the length of the elongated member comprising:
an elongated section;
an engagement section disposed at a first end of the elongated section; and
a seal assembly disposed at a second end of the elongated section;
wherein the elongated member is adapted to interface with a body of a substance delivery device.

2. The substance delivery device of claim 1, wherein the elongated member is configured to maintain a predetermined separation distance between the gripping section and the first end of the elongated member.

3. The substance delivery device of claim 1, wherein the elongated member is configured to be reciprocated through a predefined stroke length to move the seal assembly through the conduit of the body from a fully retracted position to a fully depressed position to drive a substance when present in the body out of the body through the opening.

4. The substance delivery device of claim 1 wherein the engaging member is configured to engage a digit or heel of the user's hand as the user reciprocates the elongated member.

5. The substance delivery device of claim 1 wherein the elongated member has a length that is substantially 110% or greater than a preselected overall length of the body such that the engaging member is disposed outward from an end of the body a predetermined distance when the elongated member is disposed in the fully depressed position.

6. The substance delivery device of claim 1, wherein the gripping section is disposed at an end of the body.

7. The substance delivery device of claim 1, wherein the elongated member has a length that is greater than about 120% of the preselected overall length of the body.

8. The substance delivery device of claim 1, wherein a portion of the elongated member proximate the engaging member is enlarged with respect to the rest of the elongated member for increased stability during use.

9. The substance delivery device of claim 1, wherein the elongated member comprises a tapered outer periphery such that the diameter of the elongated member increases along the length of the elongated member towards the engaging member.

10. The substance delivery device of claim 1, wherein the elongated member comprises a flared end.

11. The substance delivery device of claim 1, wherein a diameter of the engaging member has a larger diameter than the rest of the elongated member.

12. The substance delivery device of claim 1, wherein at least a portion of the engaging member is concave.

13. The substance delivery device of claim 1, wherein the engaging member is configured to conform to a user's digit during use.

14. The substance delivery system of claim 1, wherein the substance comprises a viscous fluid.

15. The substance delivery system of claim 1, wherein the substance comprises a gel.

16. The substance delivery system of claim 1, wherein the substance a viscous paste.

17. A cap assembly adapted to removably couple to a container comprising:
a body having:
a receiving portion adapted to couple to at least a portion of the container;
a retaining assembly adapted to removably couple the cap to the container when a portion of the container is disposed within the receiving portion of the cap assembly; and
a leveragable portion adapted to allow for the removal of the cap from the container, wherein the leveragable portion has a shape which allows for leverage applied there against to cause the removal of the cap from the container.

18. The cap of claim 17, wherein the leveragable portion has an outwardly contoured portion.

19. The cap of claim 17, wherein the leveragable portion further comprises frusto-conical shape in a side view.

20. The cap of claim 17, wherein the leveragable portion comprises a disc shaped member in a side view at either end of the cap.

21. The cap of claim 17, wherein the leveragable portion comprises a disc shaped member in a side view at both ends of the cap.

22. The cap of claim 17, further comprising a stop configured to prevent the tip of the substance delivery device from protruding beyond a plane defining the end of the cap.

23. A cap removal device comprising:
a cantilever portion configured to allow a cap as defined in claim 17 to be removed from the container;
a support portion configured to provide support to the cantilever portion;
wherein the cantilever portion forms with the support portion a first receiving recess, the cantilever portion also including two prongs forming a second recess therebetween such that in use when the leveragable portion of the cap is inserted in the first receiving recess and the hollow container receiving portion is inserted in the second recess, the cap can be removed from the container.

24. A substance delivery system comprising:
a substance delivery device comprising:
a body comprising:
a conduit therethrough;
an opening at a first end of the body in communication with the conduit;
an aperture disposed at a second end of the body in communication with the conduit; and
a gripping section;
an elongated member comprising:
an engagement section;
an elongated section; and
a seal assembly;
a cap comprising:
a receiving portion;
an opening disposed in one end of the receiving portion;
a remover configured to allow removal of the cap;
a cap removal device comprising:
a cantilever portion configured to allow the cap to be removed from the substance delivery device;
a support portion configured to provide support to the cantilever portion; and
a receiving recess wherein the receiving recess is formed at a juncture of the cantilever portion and the support portion.

25. The substance delivery system of 24, wherein the cantilever portion further comprises two prongs forming a second recess therebetween configured to engage the receiving portion of the cap.

26. The substance delivery system of 24, wherein the remover comprises at least one protrusion.

27. The substance delivery system of 24, wherein the remover comprises a flange.

28. The substance delivery system of 24, wherein the remover are disposed on proximal end or distal end of receiving portion.

29. The substance delivery system of claim 24 wherein the support portion is supported by means of a band member adapted to be attached to a wrist or belt or other object.

30. The substance delivery system of claim 24 wherein the support portion is adapted to be hung from an upstand.

31. The substance delivery system of 24, wherein the substance delivery system is used in veterinary applications.
